(19) United States
(12) Patent Application Publication
(10) Pub. No.: US 2012/0252765 A1
(43) Pub. Date: Oct. 4, 2012

(54) USE OF IRBESARTAN FOR THE PREPARATION OF A MEDICAMENT FOR THE PREVENTION OF HOSPITALIZATION FOR HEART FAILURE

(75) Inventors: Catherine MARCHESE, Paris (FR); Christophe GAUDIN, Saint Cloud (FR)

(73) Assignee: SANOFI, Paris (FR)

(21) Appl. No.: 13/405,896

(22) Filed: Feb. 27, 2012

Related U.S. Application Data
(63) Continuation of application No. PCT/IB2010/053904, filed on Aug. 31, 2010.

(30) Foreign Application Priority Data
Aug. 31, 2009 (FR) 0904132

(51) Int. Cl. A61K 31/4184; A61K 31/616; A61P 7/02; A61P 9/04

(52) U.S. Cl. 514/161; 514/381

(57) ABSTRACT

Use of irbesartan or one of its pharmaceutically acceptable salts for the preparation of a medicament for the prevention of hospitalization for heart failure.

This application is a continuation of International application No. PCT/IB2010/053904, filed Aug. 31, 2010, which is incorporated herein by reference in its entirety; which claims the benefit of priority of French Patent Application No. 0904132, filed Aug. 31, 2009.

The present invention relates to the use of irbesartan or pharmaceutical salts thereof for the preparation of a medicament for the prevention of hospitalization for heart failure.

Irbesartan is the 2-n-butyl-4-spirocyclopentane-1-[(2′-(tetrazol-5-yl)biphenyl-4-yl)methyl]-2-imidazolin-5-one and it is represented by the following formulae:

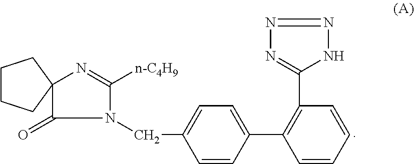

Irbesartan as well as its processes of manufacture are described in patent EP 454511. Irbesartan exist in two tautomeric forms: form A (represented above), and form B, described for example in patent EP 708103 and represented by the following formulae:

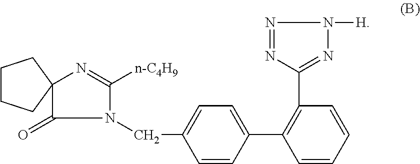

In the present specification and claims, irbesartan should be understood as meaning equally irbesartan form A or irbesartan form B, unless otherwise specified.

Irbesartan interrupts the renin-angiotensin system by means of selective blockade of the angiotensin II subtype 1 receptor, and it is an effective antihypertensive agent. It further has an excellent pharmacokinetic profile (it is cleared primarily by oxidation via cytochrome P450), is well absorbed orally, and its side effects do not differ significantly from placebo. Irbesartan has also been shown to reduce left ventricular mass in patients with hypertension, and further has a significant beneficial effect on nephropathy progression in hypertensive diabetic patients with proteinuria. In patients with heart failure, addition of irbesartan to conventional treatment, such as diuretics and ACE inhibitors, improved left ventricular ejection fraction and exercise time.

The applicant has now clinically proven that irbesartan, and in particular irbesartan form A, reduces the occurrence of hospitalization for heart failure in patients with atrial fibrillation and additional major risk factors while this was not demonstrated for other antihypertensive compounds. It was further not demonstrated for patients having atrial fibrillation or a history of atrial fibrillation.

The object of the present invention is the use of irbesartan or one of its pharmaceutically acceptable salts, and in particular irbesartan form A or one of its pharmaceutically acceptable salts, for the preparation of a medicament for the prevention of hospitalization for heart failure, notably in patients with a history of atrial fibrillation or patients having atrial fibrillation.

A further object of the present invention is the use of irbesartan or one of its pharmaceutically acceptable salts, and in particular irbesartan form A or one of its pharmaceutically acceptable salts, for the preparation of a medicament for the prevention of hospitalization for heart failure, notably in patient with a history of atrial fibrillation or patients having atrial fibrillation, said patient being further treated with at least one of the following medication:
a) clopidogrel and acetylsalicylic acid,
b) oral anticoagulant such as warfarin,
c) acetylsalicylic acid.

In the present specification, “hospitalization for heart failure” is defined to occur when a patient is admitted overnight to a hospital with symptoms or signs of heart failure and one of the following: radiological evidence of congestive heart failure, use of intravenous inotropic agents or the use of intravenous diuretics.

Examples of symptoms or signs of heart failure are listed in the following paragraphs.

The most common symptoms are shortness of breath and fatigue, but in older people, heart failure sometimes causes vague symptoms such as sleepiness, confusion, and disorientation.

Left-sided heart failure leads to fluid accumulation in the lungs, which causes shortness of breath. At first, shortness of breath occurs only during exertion, but as heart failure progresses, it occurs with less and less exertion and eventually occurs even at rest. People with left-sided heart failure also feel tired and weak when performing physical activities, because their muscles are not receiving enough blood.

A sudden accumulation of a large amount of fluid in the lungs (acute pulmonary edema) causes extreme difficulty breathing, rapid breathing, bluish skin, and feelings of restlessness, anxiety, and suffocation. Some patients have severe spasms of the airways (bronchospasms) and wheezing. Acute pulmonary oedema is a life-threatening emergency.

When the heart is severely damaged, blood clots can form because blood flow within the chambers is sluggish. Clots may break loose (becoming emboli), travel through the bloodstream, and partially or completely block an artery elsewhere in the body. If a clot blocks an artery to the brain, a stroke may result.

The term “radiological evidence of congestive heart failure” is well understood by the practitioner, who observes mainly two principal features of the chest radiograph: (1) the size and shape of the cardiac silhouette, and (2) oedema at the lung bases.

Atrial fibrillation is a common cardiac arrhythmia with the potential for serious consequences. It affects over 1% of the population and is much more common in the elderly. Over the age of 35 years, the prevalence of atrial fibrillation is 2.8%; over the age of 75 years, the prevalence is 12-16%. One of the serious outcomes associated with atrial fibrillation is stroke, which occurs at an annual rate of 4.5% in atrial fibrillation patients.

Atrial fibrillation leads to formation of thrombus in the left atrium, which can dislodge into the systemic circulation. About 15% of all strokes are directly attributable to atrial fibrillation, and in those over 80 years atrial fibrillation is the single leading cause of major stroke. About two thirds of strokes in patients with atrial fibrillation are cardioembolic. The others are due to carotid atherosclerosis, platelet emboli and hypertension. This is because the most powerful risk factors for developing atrial fibrillation are systemic hypertension and advancing age, which lead to vascular events of all kinds.

In the six placebo-controlled trials of anti-thrombotic therapy in atrial fibrillation, the annual rate of serious vascular events was 9.4% in the placebo patients, and about two thirds of these were strokes. In the placebo treated patients (n=779) in the pivotal Stroke Prevention in Atrial Fibrillation (SPAF) study, there were 25 strokes that were at least moderately disabling or fatal. In addition, there were 14 myocardial infarctions and 39 other vascular deaths. Thus, atrial fibrillation is a very powerful marker for development of stroke, myocardial infarction, congestive heart failure and other vascular events that reduce the quality and duration of life.

Another object of the invention is a pharmaceutical composition which comprises, as active principle, irbesartan or one of its pharmaceutically acceptable salts. This pharmaceutical composition comprises an effective dose of irbesartan according to the invention, or an addition salt thereof with a pharmaceutically acceptable salt, or a hydrate or solvate thereof, and at least one pharmaceutically acceptable excipient.

For their therapeutic use, irbesartan and pharmaceutically acceptable salts thereof are generally introduced into pharmaceutical compositions.

These pharmaceutical compositions contain an effective dose of irbesartan or of pharmaceutically acceptable salts thereof, and also at least one pharmaceutically acceptable excipient.

Said excipients are chosen according to pharmaceutical form and the method of administration desired, from the usual excipients which are known to those skilled in the art.

In said pharmaceutical compositions for oral, sublingual, subcutaneous, intramuscular, intravenous, topical, local, intratracheal, intranasal, transdermal or rectal administration, irbesartan, or a pharmaceutically acceptable salt thereof, can be administered in unit administration form, as a mixture with conventional pharmaceutical excipients, to animals and to humans in the cases mentioned below.

The suitable unit administration forms comprise forms for oral administration, such as tablets, soft or hard gel capsules, powders, granules and oral solutions or suspensions, sublingual, buccal, intratracheal, intraocular or intranasal forms, forms for administration by inhalation, topical, transdermal, subcutaneous, intramuscular or intravenous administration forms, rectal administration forms, and implants. For topical application, irbesartan and pharmaceutically acceptable salts thereof can be used in gels, creams, ointments or lotions.

The dose of irbesartan administered per day orally, is usually of 150 mg or 300 mg, taken most often once daily, at night.

There may be specific cases were higher or lower dosages do not depart from the context of the invention. According to the usual practice, the dosage appropriate for each patient is determined by the physician according to the method of administration, the weight, the pathology, the body surface, the cardiac output and the response of said patient.

As an example, a dosage form of irbesartan or one of its pharmaceutically acceptable salts, in the form of a tablet, can comprise the following ingredients:

| | Quantity per |
|---|---|
| Name of ingredients | tablet (mg) |
| ACTIVE INGREDIENT: | |
| Irbesartan | 150.0 mg |
| EXCIPIENTS | |
| Lactose, Monohydrate | 30.75 mg |
| Microcrystalline Cellulose | 39.00 mg |
| Pregelatinized Starch | 45.00 mg |
| Poloxamer 188 | 9.00 mg |
| Croscarmellose Sodium | 15.00 mg |
| Silicon Dioxide | 8.25 mg |
| Magnesium stearate | 3.00 mg |
| Purified Water or Water for injection | — |
| Tablet mass | 300 mg |

The instant invention also relates to a method of prevention of hospitalization for heart failure which comprises the administration to a patient of an effective dose of at least irbesartan or one of its pharmaceutically acceptable salts, and in particular irbesartan form A or one of its pharmaceutically acceptable salts.

A further object of the present invention also relates to a method of prevention of hospitalization for heart failure which comprises the administration to a patient of an effective dose of at least irbesartan or one of its pharmaceutically acceptable salts, and in particular irbesartan form A or one of its pharmaceutically acceptable salts, wherein the prevention of hospitalization consists both in a reduction of the hospitalization for heart failure, and in the duration of said hospitalization.

### BRIEF DESCRIPTION OF THE DRAWINGS

The invention is illustrated with the above data with reference to the following FIGURE:

FIG. 1 represents Kaplan Meier cumulative incidence curves of time to first hospitalization for heart failure according to the mean on-treatment analysis of 4.1 years.

Efficacy of irbesartan and its pharmaceutically acceptable salts versus placebo in patients with atrial fibrillation for the prevention of vascular events was provided via irbesartan during a partial factorial, double-blind, randomized, multi-center, placebo-controlled group trial.

I. Selection of Patients

Inclusion Criteria:

To be eligible for the trial, patients had to have all of the following: an evidence of atrial fibrillation, an evidence of high risk of vascular events, and a systolic blood pressure of at least 110 mm Hg.

The evidence of atrial fibrillation is defined as follows: patient must have either permanent, paroxysmal or persistent atrial fibrillation. Patients in atrial fibrillation at baseline (unless cardiovascular surgery was performed in the previous month) had ECG documented atrial fibrillation at the time of enrollment. Patients not in atrial fibrillation at baseline (i.e. during screening period, between the screening visit and the day of randomization) had ECG documented atrial fibrillation on two separate occasions, at least 2 weeks apart in the six months prior to randomization (and not within one month of cardiovascular surgery).

Atrial fibrillation was documented by routine ECG, rhythm strip, Holter ECG or pacemaker atrial lead telemetry electrogram. In the case of the Holter ECG or pacemaker telemetry, the duration of atrial fibrillation was to be at least one minute.

The evidence of high risk of vascular events is defined as follows: at least one of the following risk criteria must be present:

a) age 75 years or greater;

b) on treatment for systemic hypertension;

c) prior stroke, transcient ischemic attack or non-central nervous system systemic embolus;

d) left ventricular dysfunction with left ventricular ejection fraction estimated by echocardiogram or angiogram (radionuclide or contrast) to be <45%;

e) peripheral vascular disease (previous peripheral artery revascularization, limb and foot amputation, or the combination of current intermittent claudication and ankle arm systolic blood pressure ratio<0.9);

f) age 55 to 74 years and either:
f1) diabetes mellitus requiring drug therapy, or
f2) documented previous myocardial infarction or documented coronary artery disease.

Exclusion Criteria

Patients were excluded from the trial if any of the following was present:

a) requirement for clopidogrel (such as recent coronary stent procedure);

b) requirement for oral anticoagulant (such as prosthetic mechanical heart valve);

c) prior intolerance to acetylsalicylic acid or clopidogrel;

d) documented peptic ulcer disease within the previous 6 months;

e) prior intracerebral hemorrhage;

f) significant thrombocytopenia (platelet count<50×10⁹/L);

g) psychosocial reason making study participation impractical;

h) geographic reason making study participation impractical;

i) ongoing alcohol abuse;

j) mitral stenosis;

k) pregnant or nursing woman or woman of child bearing potential and not on effective birth control for at least one month prior to start of study or not willing to continue on birth control for duration of study;

l) severe comorbid condition such that the patient is not expected to survive 6 months;

m) patient currently receiving an investigational pharmacologic agent;

n) requirement for chronic (>3 months) non-COX-2 inhibitor NSAID (Non Steroidal Anti-Inflammatory Drug) therapy unless willing to be enrolled in the trial;

o) already receiving an angiotensin receptor blocking agent (unless willing and able to be switched to another antihypertensive agent);

p) previous intolerance to an angiotensin receptor blocking agent;

q) proven indication for an angiotensin receptor blocking agent (unless willing and able to substitute an angiotensin converting enzyme inhibitor).

II. Duration and Treatment

Patients have been enrolled for over 4 years. Study drug treatment units (placebo or irbesartan) were such that each patient took two tablets of either placebo or 150 mg irbesartan form A, once daily, at night. After an up-titration period of 2 weeks, all patients were up-titrated to 300 mg. They took two tablets of either placebo or 150 mg irbesartan form A, once daily, at night.

III. Results

Results were calculated using non-parametric Kaplan-Meier estimate.

Cox's proportional hazard model was used to estimate the hazard ratio also called relative risk.

Relative risk (RR) is the ratio between the risk of having a hospitalization for heart failure for patients treated with irbesartan and the risk of having a hospitalization for heart failure for patients treated with placebo.

The percentage of decrease of an event is calculated as follow:

x=1−RR.

From the 9016 patients included in the trial, 4518 randomized patients were treated with irbesartan, 4498 patients were treated with placebo. The study showed a significant reduction in the risk of heart failure hospitalization (3.2% per year in the placebo group versus 2.7% with irbesartan) by 14% (p=0.018), corresponding to 551 hospitalization for heart failure events reported in the placebo group versus 482 in the group treated with irbesartan.

Calculated relative risk was equal to 0.86, i.e. a decrease of the relative risk of hospitalization for heart failure of 14% with a p value of 0.018, and a confidence interval of 0.76-0.98.

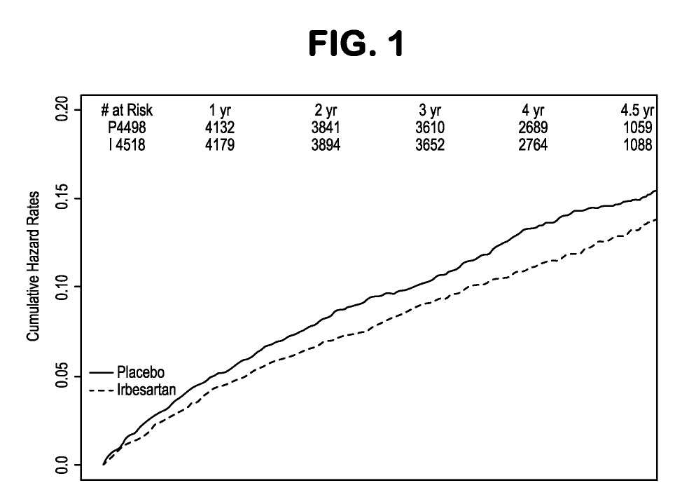

FIG. 1 demonstrates a separation of both groups already early into the study and continuously diverge throughout the observation period.

Post Hoc analysis indicated a significant reduction in the risk of the composite of stroke, non-CVS embolism and transient ischemic attacks (3.4% per year in the placebo group versus 2.9% with irbesartan) by 13% (p=0.02).

The number of stays in hospital (4059 placebo versus 3817 irbesartan, p=0.004) were significantly reduced, as well as days hospitalized for cardiovascular reasons were significantly reduced (39941 placebo versus 36480 irbesartan p=0.0001), corresponding to a decrease of 8.7%.

What is claimed is:

1. A method of preventing hospitalization for heart failure in a patient, comprising administering to said patient an effective dose of irbesartan or a pharmaceutically acceptable salt thereof, wherein said patient has a history of atrial fibrillation or a current atrial fibrillation.

2. The method according to claim 1, wherein the patient has at least the three following factors:
an evidence of atrial fibrillation,
an evidence of high risk of vascular events, and
a systolic blood pressure of at least 110 mm Hg.

3. The method according to claim 2, wherein the patient has a left ventricular dysfunction with left ventricular ejection fraction estimated by echocardiogram or angiogram (radionuclide or contrast) to be <45%.

4. The method according to claim 2, wherein the patient further has at least one of the following risk factors:
a) age 75 years or greater;
b) on treatment for systemic hypertension;
c) prior stroke, transcient ischemic attack or non-central nervous system systemic embolus;
d) left ventricular dysfunction with left ventricular ejection fraction estimated by echocardiogram or angiogram (radionuclide or contrast) to be <45%;
e) peripheral vascular disease (previous peripheral artery revascularization, limb and foot amputation, or the combination of current intermittent claudication and ankle arm systolic blood pressure ratio<0.9); or
f) age 55 to 74 years and either:
f1) diabetes mellitus requiring drug therapy, or
f2) documented previous myocardial infarction or documented coronary artery disease.

5. The method according to claim 1 wherein irbesartan is irbesartan form A.

6. The method according to claim 1 wherein irbesartan is administered at a dose up to 300 mg/day.

7. The method according to claim 5 wherein irbesartan is administered at a dose up to 300 mg/day.

8. The method according to claim 1 wherein the patient is also treated with at least one of the following medication:
a) clopidogrel and acetylsalicylic acid,
b) oral anticoagulant,
c) acetylsalicylic acid.

9. The method according to claim 5 wherein the patient is also treated with at least one of the following medication:
d) clopidogrel and acetylsalicylic acid,
e) oral anticoagulant,
f) acetylsalicylic acid.

10. The method according to claim 7 wherein the patient is also treated with at least one of the following medication:
g) clopidogrel and acetylsalicylic acid,
h) oral anticoagulant,
i) acetylsalicylic acid.

11. The method according to claim 1 wherein the prevention of hospitalization for heart failure is a decrease of 14% in the risk of heart failure hospitalization.

12. The method according to claim 5 wherein the prevention of hospitalization for heart failure is a decrease of 14% in the risk of heart failure hospitalization.

13. The method according to claim 7 wherein the prevention of hospitalization for heart failure is a decrease of 14% in the risk of heart failure hospitalization.

14. The method according to claim 10 wherein the prevention of hospitalization for heart failure is a decrease of 14% in the risk of heart failure hospitalization.

15. The method according to claim 1 wherein the prevention of hospitalization for heart failure is a decrease of 8.7% in the number of days of hospitalization for cardiovascular reasons.

16. The method according to claim 5 wherein the prevention of hospitalization for heart failure is a decrease of 8.7% in the number of days of hospitalization for cardiovascular reasons.

17. The method according to claim 7 wherein the prevention of hospitalization for heart failure is a decrease of 8.7% in the number of days of hospitalization for cardiovascular reasons.

18. The method according to claim 10 wherein the prevention of hospitalization for heart failure is a decrease of 8.7% in the number of days of hospitalization for cardiovascular reasons.

19. The method according to claim 14 wherein the prevention of hospitalization for heart failure is a decrease of 8.7% in the number of days of hospitalization for cardiovascular reasons.
